(19) United States
(12) Patent Application Publication
(10) Pub. No.: US 2008/0038768 A1
(43) Pub. Date: Feb. 14, 2008

(54) Assay for ubiquitin mediated proteolysis

(76) Inventors: Ireos Filipuzzi, Basel (CH); Bernd Gerhartz, Lorrach (DE); Lukas Leder, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/501,508

(22) Filed: Aug. 8, 2006

(51) Int. Cl. C12Q 1/37

(52) U.S. Cl. 435/23

(57) ABSTRACT

The present invention provides methods and compositions useful in the screening for agents that modulate activity of a deubiquitinating enzyme. In one embodiment, the invention relates to methods for measuring a deubiquitination activity comprising combining a deubiquitinating enzyme (DUB) with a substrate comprising a ubiquitin moiety and a fluorescently labeled compound under conditions allowing for deubiquitinating activity and measuring an altered fluorescence polarization and/or fluorescence lifetime of the released fluorescently labeled compound The invention also relates to a substrate library comprising peptides of the formula (Z)ₐ(X)ₘK(X)ₙ(Z)_{b} useful e.g. for the determination of the substrate specificity of a deubiquitinating enzyme.

### FIELD OF THE INVENTION

The invention relates to the field of ubiquitin mediated proteolysis. In particular, the invention relates to methods and compositions for screening for agents that modulate activity of a deubiquitinating enzyme.

### BACKGROUND OF THE INVENTION

The intracellular turnover of proteins is tightly regulated through ubiquitination and deubiquitination where ubiquitin (Ub), an 8.6 kDa highly conserved protein, represents the trigger for degradation. In addition, the posttranslational modification of proteins by the large number of ubiquitin-like proteins are regulated in a similar manner. Generally, all these modification reactions share the same chemistry. This tagging reaction involves three sequential enzyme-catalyzed reactions that ultimately ligate the C-terminal Gly of Ubiquitin onto ε-amines of Lys residues of the substrate protein. A polyUb chain is then formed on the protein by the ligation of additional Ub monomers in successive rounds of ubiquitination. These Ub molecules are added to specific Lys residues of the proximal Ub in the propagating polyUb chain. Proteins with long polyUb chains are recognized by the 26S proteasome complex for proteolytic degradation and recycling of intact Ub monomers. Alternative, the polyUB chains can be specifically removed by deubiquitinating enzymes (DUBs) and by this the protein is rescued from degradation process. Recently, important biological roles have been discovered for DUBs. For example, the human Unph gene encodes a deubiquitinating enzyme whose over-expression leads to oncogenic transformation of NIH3T3 cells; the tre-2 oncogene is structurally related to Unph and also encodes a deubiquitinating enzyme; DUB1 and DUB2 are a subfamily of cytokine-inducible, immediate early genes that encode a deubiquitinating enzyme with growth regulatory activities; UCH-L1 is not only involved in neural development but also in the differentiation of a lymphoblastic leukemia cell line, Reh; and the Drosophilia fat facets gene encodes a deubiquitinating enzyme which is required for eye development in the fly. USP2 (ubiquitin-specific protease-2) is found to be over-expressed in prostate cancer. It deubiquitinates fatty acid synthase (FAS), a protein which overexpression is correlated to biologically aggressive human tumors. Decreased deubiquitination by functional inhibition of USP2 results in a lower level of FAS and enhanced apoptosis.

There are numerous deubiquitinating enzymes known in eukaryotic cells that catalyse the hydrolysis of the peptide bond at G76 of the ubiquitin domain Ub¹⁻⁷²-Leu⁷³-Arg⁷⁴-Gly⁷⁵-Gly⁷⁶-X, where X is a protein or another molecule of Ub. The DUBs were grouped into two distinct families of cysteine proteases; ubiquitin-specific proteases (UBP) and ubiquitin C-terminal hydrolases (UCH). Most recently, with the identification of unconventional deubiquitinating enzymes (DUBs), this classification was revised and new families are now emerging. Whereas the UCH family comprises only 4 members in human, the USP family is highly diverse with more than 60 mammalian members, and the total number of DUBs including the DUB-like enzymes is now approaching 110. These families share two regions of similarity within a core domain, a region that contains the conserved cysteine which are probably implicated in the catalytic mechanism (cysteine box) and a C-terminal region that contains two conserved residues, histidine and aspartate. The core domain is surrounded by divergent sequences, most commonly at the N-terminus end and the functions of these divergent sequences remain unclear. Despite differences in physiological role and size, all deubiquitinating enzymes appear to hydrolyze their substrates through a common chemical mechanism involving the binding of Ub¹⁻⁷²-Leu⁷³-Arg⁷⁴-Gly⁷⁵-Gly⁷⁶-X, nucleophilic attack of the thiol moiety of the active site Cys residue on the carbonyl carbon of Gly⁷⁶, formation of an acyl-enzyme intermediate, Ub¹⁻⁷⁵-NHCH₂C(O)S-E, and hydrolysis of this intermediate to liberate free enzyme and Ub.

Their biological roles makes the DUBs interesting targets for the pharmaceutical industry for the search for pharmacologically active compounds useful in the treatment of various diseases. For instance, UCH-L3 and USP-2 are believed to inhibit the degradation of many tumor growth promoting proteins by counteracting the ubiquitination. Therefore inhibitors of these DUB enzymes should lead to enhanced degradation of oncoproteins and may lead to an early stop of tumor growth. In order to identify agents that modulate DUB activity, there is a need for rapid and easy DUB assays. Such assay will be highly useful for identifying compounds which modulate DUB protein activity.

A number of deubiquitination assays is known in the art. Deubiquitination assays are described for instance in WO04/020458. For many of these assays naturally occurring substrates have been used for designing in-vitro substrates. Deubiquitinating enzymes remove C-terminal attached peptides/proteins from ubiquitin. These extensions can be fusion proteins where the α-amino group of the N-terminus is bound to the C-terminus. In another kind of substrates, the α-amino group of a lysine residue in a peptide/protein is linked via an isopeptide bond to the C-terminus of ubiquitin. These different types of naturally occurring substrates have been used for designing in-vitro substrates in the past. Natural occurring fusion proteins like ubiquitin-L40 or polyubiquitin as well as designed fusion proteins are incubated with DUBs and the change of the molecular weight was measured upon release of one fusion partner. The detection method was based on a separation of products like HPLC or SDS-PAGE. Signal enhancing systems like radioactive labeling or epitope mapping has been used to improve the detection limit. Similarly, deubiquitination assays are known for substrates comprising an isopeptide bond as DUB-specific cleavage side which are mainly based on branched polyubiquitin chains. These polyubiquitin chains can be formed by using the ubiquitin ligase enzymes E1 and E2-25K. Using the same method, lysine or acetylated lysine had been attached to the C-terminus of ubiquitin. Cleavage of the isopeptide bond was followed by monitoring separation via HPLC. In another substrate commercially available from several suppliers, 7-amido-4-methylcoumarin (AMC) is linked to the C-terminus of ubiquitin. Release of AMC from ubiquitin results in a fluorescent signal. However, Ub-AMC has two critical disadvantages when used in screens for DUB inhibitors: First, AMC has an excitation wavelength in the UV range. Exciting at 260 nm is known to excite a significant number of screening compounds and thus will generate a large fraction of false positives. Second, AMC is covalently attached at the C-terminal COOH of Ub and not via a ε-NH₂ group as found under physiological conditions. Thus the artificial Ub-AMC substrate might not be optimal for the identification of specific inhibitors of members of the DUB family.

The present invention now provides deubiquitination assays that overcome shortcomings of the current assays and, which are particularly suitable for use in miniaturized rapid high throughput screening deubiquitination assays.

### SUMMARY OF THE INVENTION

In a first aspect, the present invention provides a method for measuring a deubiquitination activity comprising combining a deubiquitinating enzyme (DUB) with a substrate comprising a ubiquitin moiety and a fluorescently labeled compound under conditions allowing for deubiquitinating activity and measuring an altered fluorescence polarization of the released fluorescently labeled compound. In a preferred embodiment, the fluorescently labeled compound is an amino acid or a peptide containing a fluorophore which is attached to the C-terminus of a ubiquitin moiety via a peptide, or preferably, via an isopeptide bond.

In another aspect, the present invention provides screening methods for a modulating agent of a DUB comprising assaying deubiquitination activity according to a deubiquitination assay in accordance with the present invention. In a preferred embodiment, the method is performed in a high throughput screening platform.

In another aspect, the present invention provides a substrate library comprising peptides of the formula (Z)ₐ(X)ₘK(X)ₙ(Z)_{b} wherein X can be any amino acid except Lys wherein Z is distinct amino acid which is used to increase solubility preferably a charged amino acid, more preferably an Arg and wherein a and b are independently an integer from 0 to 5, and n and m are independently an integer from 0 to 20 provided that not both n and m are 0 and wherein K is attached to the C-terminus of a ubiquitin moiety via an isopeptide bond. In one embodiment the substrate library is used to identify the substrate specificity of a DUB comprising reacting the DUB with a substrate library.

### DESCRIPTION OF THE DRAWINGS

FIG. 1: “Enzymatic coupling of fluorescent Lys-derivatives” principle.

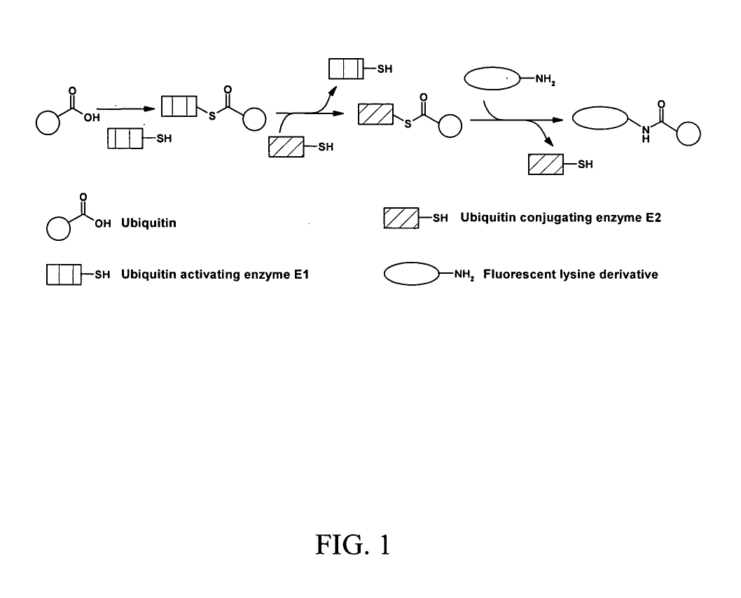

FIG. 2: USP-2 and UCH-L3 reaction time course. Enzyme concentration was varied as indicated at a constant substrate concentration of 20 nM. The reaction was monitored over time as decrease in fluorescence polarization of NusA-Ub-K48R-lysine-TAMRA upon cleavage by UCH-L3 (top) and USP-2 (bottom).

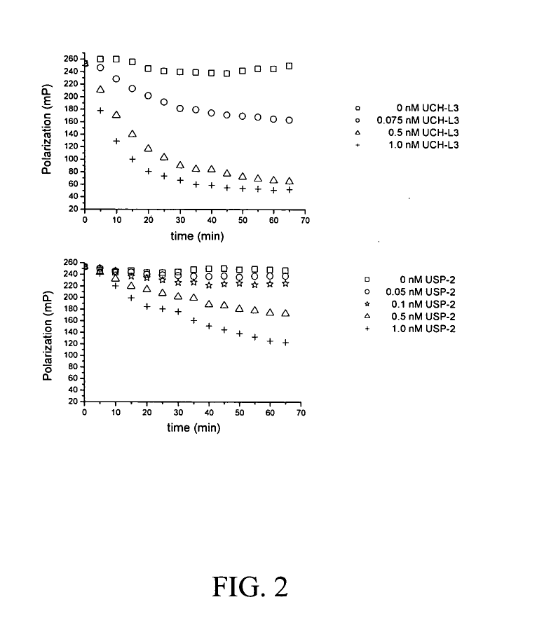

FIG. 3: Substrate titration. Different concentrations of NusA-Ub-K48R-lysine-TAMRA were incubated with 0.5 nM UCH-L3 (top) or 10 nM of USP-2 (bottom) and the reactions were monitored as decreasing fluorescence polarization. The resulting data were transformed to express the formed product as a function of time (Pt). Rates of initial velocity were then calculated and plotted against substrate concentration to determine Vmax and KM.

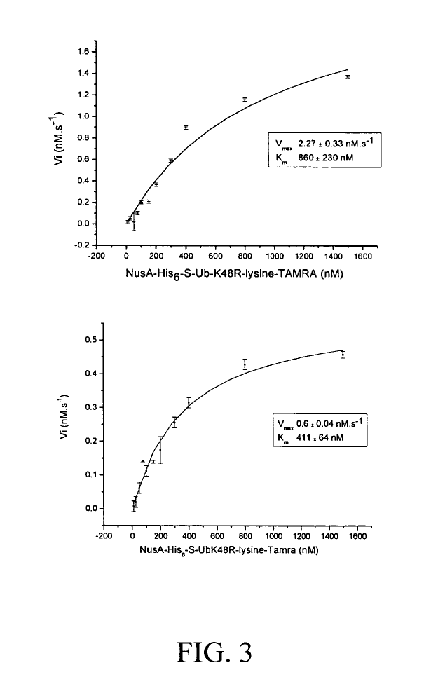

FIG. 4: Simulation of competitive inhibitors with various Ki values. The effect of potential inhibitors with Ki values from 100 nM to 20 μM are simulated at 20 μM screening concentration under standard assay conditions.

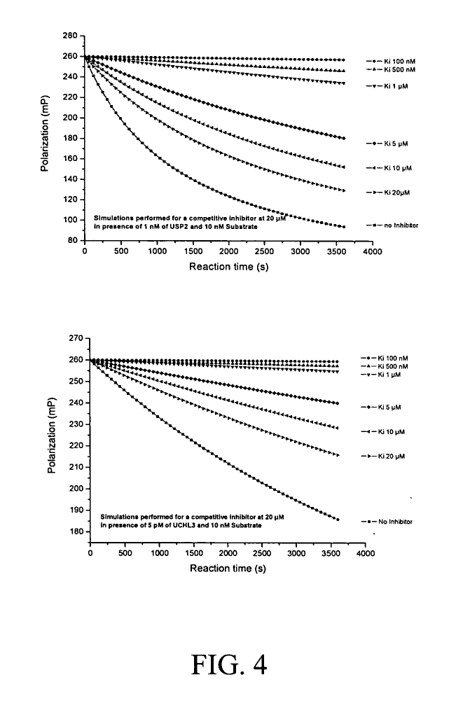

### DETAILED DESCRIPTION OF THE INVENTION

In a first aspect, the present invention provides assays for measuring the activity of deubiquitinating enzymes (DUBs) which are based on a change in fluorescence polarization and/or fluorescence lifetime upon release of a fluorescently labeled compound of a suitable substrate by a deubiquitinating enzyme. Accordingly, the present invention provides a method for measuring a deubiquitination activity comprising combining a deubiquitinating enzyme (DUB) with a substrate comprising a ubiquitin moiety and a fluorescently labeled compound under conditions allowing for deubiquitinating activity and measuring an altered fluorescence polarization and/or fluorescence lifetime of the released fluorescently labeled compound.

The term “deubiquitinating enzyme” within the context of the present invention refers to any protein showing deubiquitinating activity, i.e. the release or cleavage of a ubiquitin moiety from a ubiquitin complex. The term “deubiquitinating enzyme” within the context of the present invention refers to any protein showing deubiquitinating activity, i.e. the release or cleavage of a ubiquitin moiety from a ubiquitin complex. A deubiquitinating enzymes is typically a cysteine protease and may be classified into subgroups as ubiquitin-specific proteases (UBP) and ubiquitin C-terminal hydrolases (UCH). Examples of deubiquitinating enzymes include for instance USP5, USP6, USP4, USP8, USP13, USP2, USP11, USP14, USP7, USP9X, USP10, USP1, USP12, USP16, USP15, USP17, USP19, USP20, USP3, USP9Y, USP18, USP21, USP22, USP33, USP29, USP25, USP36, USP32, USP26, USP24, USP42, USP46, USP37, USP28, USP47, USP38, USP44, USP50, USP35, USP30, Mername-AA088peptidase, Mername-AA091 peptidase, USP45, USP51, USP34, USP48, USP40, USP31, Mername-AA129peptidase, USP49, USP17-like peptidase, USP54, USP53, USP39, UCH-L1, UCH-L3, UCH-BAP1, UCH-UCH37, Cezanne deubiquitinating peptidase, Cezanne2, tumor necrosis factor alpha-induced protein 3, TRABID protein, VCP(p97)/p47-interacting protein, otubain1, otubain2, CylD protein, SENP1 peptidase, SENP3 peptidase, SENP6 peptidase, SENP2 peptidase, SENP5peptidase, SENP7peptidase, SENP8peptidase, SENP4peptidase, Poh1 peptidase, Jab1/MPN domain metalloenzyme, Mername-AA 165 peptidase, Mername-AA 166 peptidase, Mername-AA 167 peptidase, Mername-AA168 protein, COP9 signalosome subunit6, 26S proteasome non-ATPase regulatory subunit7, eukaryotic translation initiation factor3 subunit5, IFP38 peptidase homologue. In a particularly preferred embodiment, the DUB is UCH-L3 and USP-2 or a functionally active variant thereof. The UCH-L3 and USP-2 preferably comprise an amino acid sequence set forth in the “Ref-Seq” repository (USP2: NM_{—}004205 and NM_{—}171997, UCH-L3: NM_{—}006002). Also encompassed within the scope of the present invention are functionally active variants of the DUBs. Such variants have an overall sequence similarity with the amino acid sequence of the particular DUB of greater than about 80%, more preferably greater than about 85%, even more preferably greater than about 90% and most preferably greater than 93%. In some embodiments the sequence identity will be as high as about 95 to 98 or 99%. In some embodiments 1, 2, 3, 4 or 5 amino acids of the native DUB may be replaced with another amino acid. Such a replacement may be conservative, i.e. one amino acid is replaced with another amino acid with similar physico-chemical properties. DUB variants also include chemically modified DUBs or biologically active fragments of the DUBs, i.e. a fragment retaining deubiquitinating activity, e.g. a fragment comprising the catalytic site of the DUB.

As used herein, the terms “ubiquitin” or “ubiquitin moiety” refers to any polypeptide comprising a ubiquitin polypeptide or a polypeptide associated with a biological activity of a deubiquitinating enzyme, a ubiquitin conjugating or a ubiquitin ligating enzyme. In a preferred embodiment, the ubiquitin moiety is the human 76 amino acid polypeptide (NM_{—}018955). In a preferred embodiment, specific lysine residues of the ubiquitin moiety are replaced with a conservative amino acid such as for instance by an arginine residue. Methods for replacement of amino acids are known and include for instance site directed mutagenesis or PCR based methods. In a preferred embodiment, the lysine 48 of the human ubiquitin has been mutated to arginine (Ub-K48R). Such ubiquitin molecules may be used in assays according to the present invention in order to avoid polyubiquitination. The terms also encompass ubiquitin-like polypeptides such as for instance the polypeptides set forth in Genbank entry numbers: NM_{—}006156 (NEDD8); NM_{—}003352 (SUMO-1, aka, UBL1); XM_{—}048691 35 (SUMO-1, aka, UBL1); NM_{—}006936 (smt3a); XM_{—}009805 (smt3a); XM_{—}095400 (smt3b); NM_{—}006937; (smt3b); XM 041583 (smt3b); NM_{—}015783 (ISG15); or NM 005101 (ISG15). The ubiquitin polypeptides according to the present invention can be prepared using the methods and sequences known in the art, or described herein below in the examples. The ubiquitin moiety may have a short peptide attached as described below, but may also be linked to one or more further ubiquitin moieties or be attached to a protein or be part of a fusion protein.

The terms peptide, polypeptide and protein are used interchangeably in context of this application and refer to molecule comprising at least two amino acids linked by a peptide bond.

In accordance with one aspect of the present invention, fluorescently labeled substrates for DUB enzymatic reactions are provided. The term “substrate” in the context of the present application includes any molecule which is recognized by a DUB or can be subject of a deubiquitination reaction. The substrates used in accordance with the present invention preferably comprise a ubiquitin moiety and a fluorescently labeled compound. The substrate may also comprise two or more ubiquitin moieties. In one preferred embodiment, the ubiquitin moiety of the substrate is part of a fusion protein, in a particularly preferred embodiment the ubiquitin moiety is fused with a large polypeptide, which is known to be highly soluble and/or enhances expression in hosts like E. coli, such as e.g. Trx, NusA, MBP, GST. In another preferred embodiment, the ubiquitin moiety comprises no lysine residue that is implicated in the formation of polyubiquitin chains, such as e.g. human Ub-K48R. The substrate comprises a fluorescently labeled compound which is preferably attached to the ubiquitin moiety, more preferably to the C-terminus of the ubiquitin. As used in the context of the present invention, the term “fluorescently labeled compound” refers to a compound comprising a fluorophore which is suitable for measuring fluorescence polarization and/or fluorescence lifetime. The fluorescently labeled compound is attached to a ubiquitin moiety or is part of a ubiquitin moiety in the substrate prior to the deubiquitination reaction. Preferably, the fluorescently labeled compound is amino acid or a peptide having a suitable fluorophore attached. The fluorescently labeled amino acid or a peptide can be prepared by methods known in the art, e.g. using site specific labeling of suitable amino acids as described herein below. The amino acid residue or peptide is linked via a peptide or, more preferably via an isopeptide bond, to a ubiquitin moiety, preferably to the C-terminus of the ubiquitin moiety. Upon cleavage of the substrate by the DUB, the fluorescently labeled compound is released from the substrate.

In one preferred embodiment, a C-terminally extended ubiquitin is provided as substrate, i.e. the C-terminus is extended by one or more amino acid residues. The attached amino acid residue or peptide is fluorescently labeled with a suitable fluorophore. The attached amino acid residue(s) are preferably linked to the C-terminus of the ubiquitin moiety via a peptide bond which is cleaved by the DUB thereby releasing the C-terminal extension. Preferably, such an extension comprises at least 1 and not more than 20, more preferably not more than 10 and most preferably not more than 5 amino acid residues. The peptide is released upon cleavage be the DUB. Any suitable amino acid may be used (in particular one of the 20 standard amino acids used be biological organisms), in a preferred embodiment the extension does not contain a lysine residue.

The fluorescently labeled compound can be attached to the substrate using the natural ubiquitination pathway. The fluorescently labeled compound, e.g. a lysine or lysine derivative containing a fluorophore or a short peptide containing a lysine or lysine derivative and a fluorophore can be enzymatically conjugated (i.e. attached) to a ubiquitin moiety. In this approach, a ubiquitin activating enzyme, such as E1, and a ubiquitin conjugating enzyme, such as E2, is employed to attach the fluorescently labeled compound to a ubiquitin moiety. Optionally, a ubiquitin ligating enzyme, such as E3, may be used in addition. This step might be required if larger peptides or proteins are attached to the ubiquitin moiety, for lysine or lysine derivatives or short peptides comprising a lysine or lysine derivative a ubiquitin ligase is normally not required. Methods for enzymatically attach lysine or lysine derivatives or peptides comprising a lysine or a lysine derivative to ubiquitin are described herein below.

The fluorescently labeled compound preferably comprises an epsilon-amino group, e.g. a lysine or a lysine derivative (lysine derivatives include e.g. ornithine, cadaverine). In one embodiment, the fluorescent lysine or lysine derivative is part of ubiquitin. In another, particularly preferred embodiment, the fluorescently labeled compound consists of the lysine or lysine derivative with a fluorophore attached. In another preferred embodiment, the lysine is part of a short peptide comprising a fluorophore. The fluorescent lysine or lysine derivative or the peptide comprising the fluorescent lysine or lysine derivative is preferably attached via an ε-NH₂ to the C-terminus of the ubiquitin moiety.

The peptide has preferably less than 50 amino acid residues, more preferably less than 20 amino acid. In one preferred embodiment, peptide has 4 to 16 amino acid residues. The peptide has preferably less than 50 amino acid residues, more preferably less than 20 amino acid. In one preferred embodiment, peptide has 4 to 16 amino acid residues. In one preferred embodiment, the peptide is a peptide of formula (Z)ₐ(X)ₘK(X)ₙ(Z)_{b} wherein X can be any amino acid except Lys wherein Z is distinct amino acid which is used to increase solubility preferably a charged amino acid, more preferably an Arg and wherein a and b are independently an integer from 0 to 5, preferably from 1 to 3, more preferably 2, and n and m are independently an integer from 0 to 20, preferably from 1 to 10, more preferably from 2 to 6 provided that not both n and m are 0.

The fluorescently labeled compound is advantageously linked to the ubiquitin moiety via a peptide or isopeptide bond. In the deubiquitination reaction, the DUB cleaves the peptide or isopeptide bond and free fluorescently labeled compound is released. Due to the changed molecular mass of the free fluorescently labeled compound upon cleavage a shift in fluorescent polarization can be measured. Alternatively, cleavage of the fluorescently labeled compound leads to altered physico-chemical environment for the fluorophore which can result in altered fluorescent lifetime. A higher dynamic range of fluorescent polarization and/or fluorescent lifetime leads to more robust read-out of the assay. The dynamic range of fluorescence polarization and fluorescent lifetime is dependent on the ratio of the molecular mass of the substrate, or physico-chemical environment of the fluorophore, respectively, comprising the ubiquitin moiety and the fluorescently labeled compound with the free fluorescently labeled compound. Thus, in order to obtain a high dynamic range of fluorescence polarization and/or fluorescent lifetime a substrate with high molecular mass is used (e.g. a ubiquitin moiety fused to a protein as described herein_below in the examples) and a small fluorescently labeled compound (e.g. a lysine derivative comprising a fluorophore or a short peptide comprising a fluorophore). In a preferred embodiment, the ratio of the molecular mass of the substrate with bound fluorescently labeled compound to the free fluorescently labeled compound is at least 5, preferably at least 5-10, more preferably at least 10-100. In another preferred embodiment, the substrate comprises a ubiquitin moiety large tags (e.g. Trx, NusA, MBP, GST) fused to the N-terminus of the ubiquitin moiety and/or a small fluorescently labeled compound such as a fluorescently labeled lysine or lysine derivative.

Any fluorophore which is suitable for measuring fluorescence polarization upon cleavage of the fluorescently labeled compound by ubiquitin may be used in accordance with the present invention. Suitable fluorophores include, but are not limited to TAMRA, Cy5 or MR121. In a preferred embodiment, the fluorophore is TAMRA.

In a one aspect of the present invention, the deubiquitinating assays of the present invention are used in the screening for agents which modulate DUB activity. Accordingly, the present invention provides screening methods for identifying a modulating agent of a deubiquitinating enzyme (DUB) comprising combining a candidate agent with a DUB and with a suitable fluorescently labeled DUB substrate under conditions allowing for deubiquitinating activity and measuring the activity of the DUB, wherein said activity is measured by fluorescence polarization and/or fluorescence lifetime.

A “modulating agent” within the context of the present invention is a compound which, when brought under suitable conditions in contact with a DUB, increases or decreases the biological activity of the DUB. Accordingly, a “modulator” is either an inhibitor of the biological activity of a DUB or a stimulator of the biological activity of a DUB.

By the term “candidate agent” refers to any candidate molecule, e.g. a protein or peptide or polypeptide, antibody, low molecular weight molecule or polynucleotide which are to be tested for the ability to modulate the biological activity of a DUB agent.

Candidate agents encompass numerous chemical classes. In a preferred embodiment, the candidate agents are low molecular weight compounds, particularly organic low molecular weight compounds, comprising functional groups necessary for structural interaction with proteins, particularly hydrogen bonding, and typically include an amine, carbonyl, hydroxyl or carboxyl group, preferably at least two of the functional chemical groups. Candidate agents may be obtained from a wide variety of sources, as will be appreciated by the skilled person, including libraries of synthetic or natural compounds. The present invention provides method for rapid and easy screening any library of candidate modulators, including the wide variety of known combinatorial chemistry-type libraries. Methods for generating new compounds for compound libraries are known and discussed for instance in WO 94/24314.

In a preferred embodiment, the candidate agent is a low molecular weight compound (e.g., organic or inorganic molecules which are less than about 2 kDa in molecular weight, more preferably less than about 1 kDa in molecular weight, and/or are able to cross the blood-brain barrier) which modulates, i.e. inhibits or stimulates, the biological activity of a DUB.

In a preferred embodiment, the candidate agents are peptides. The peptides comprise preferably from about 2 to about 50 amino acid residues, more preferably from about 5 to about 30 amino acids, and particularly preferred from about 8 to about 20 amino acid residues. The peptides may be digests of naturally occurring proteins or chemically synthesized random peptides.

In another preferred embodiment, the candidate agent is an antibody. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. As appreciated by the skilled person, various procedures known in the art may be used for the production the different kinds of antibodies.

The significant differences of fluorescence polarization and/or fluorescent lifetime signals between non-cleaved and cleaved substrate allow the using the DUB screening assay of the present invention in a miniaturized high throughput screening (HTS) assay. Such HTS assays are typically carried out in a microtiter plate in small volume and allow to run a large number of the DUB assays in parallel at the same time under the same conditions. HTS assay are particularly useful for the rapid screening of a large number of compounds, e.g. a compound library as described above. A number of HTS platforms are known and described for instance in Drug Discov Today. 2003 Nov. 15; 8(22):1035-4. by Gribbon and Sewing. Accordingly, the present invention provides deubiquitination assays based on fluorescence polarization and/or fluorescent lifetime as herein described suitable for using in a High Throughput Screening (HTS) platform.

In another aspect, the present invention provides methods to identify the substrate specificity of any DUB. For this purpose a substrate library of peptides of the formula (Z)ₐ(X)ₘK(X)ₙ(Z)_{b}. X is randomized and can be any amino acid except Lys, The Z residues at the N- and C-terminus are distinct amino acids, which are used to increase solubility, normalize the library, and can be labeled by a biotin. The biotin can be used to purify the library and to purify the peptide. During the incubation with a distinct DUB using short incubation times and low concentrations of the particular DUB, the DUB will release peptides from ubiquitin according to the substrate specificity. Substrates displaying the highest k_{cat}/Kₘ will be preferentially released. After a separation of released peptides from the uncleaved library, the preferred substrates will be found in highest concentration among the released peptides. Using MS-MS analysis, these peptides can be identified. In an other approach by sequencing the released peptides together from the N-terminus, the concentration of the different amino acids in each position will reflect the specificity of the particular DUB at the particular subside position. The substrate specificity identifies the specific substrate recognition pattern. The identified sequence can be used to identify natural substrates of the different DUBs by using bioinformatics methods.

Having generally described this invention, a further understanding can be obtained by reference to certain specific examples which are provided herein for purposes of illustration only, and are not intended to be limiting unless otherwise specified.

### EXAMPLE 1

### Substrate Labeling and Testing

### 1.1 Cloning of the Expression Constructs

For cloning of ubiquitin (UBB, Swiss Prot P02248) and the ubiquitin conjugating enzymes Cdc34 (UBC3, Swiss Prot P49427), E2-25K (HIP2, Swiss Prot P27924), Rad6B (UBE2B, Swiss Prot P23576) and UbcH10 (UBE2C, Swiss Prot 000762) a proprietary cDNA library was used. This library was created by reverse transcription of a total RNA preparation of human umbilical vein endothelial cells (HUVEC), whereby the RNA preparation was incubated with a (dT)₂₀ oligonucleotide and 80 U M-MLV reverse transcriptase (Promega, Madison Wis., USA) and 100 U RNase inhibitor (RNasin, Madison Wis., USA) for 30 min at 50° C. and for 5 min at 99° C. The inserts of the different proteins were amplified from the cDNA by a method called “sticky end PCR”. The PCR reaction was performed with Pfu polymerase (Promega, Madison Wis., USA) in 30 cycles with 15 sec at 95° C., 15 sec at 55° C. and 30 sec at 72° C. The annealed PCR products containing the BamHI and NotI restriction sites with sticky ends were then directly ligated with T4 ligase (Invitrogen, Carlsbad Calif., USA) into three different E. coli pET expression vectors (Novagen, Darmstadt, Germany). These vectors had been previously digested with BamHI and NotI and were derived from pET30a with just a N-terminal hexahistidine (His₆) tag, from pET32 with N-terminal thioredoxin (Trx) tag and from pET43.1 with a N-terminal NusA tag. In all three expression plasmids the existing enterokinase cleavage site was replaced by a PreScission protease cleavage site (L-E-V-L-F-Q-G-P) just upstream of the BamHI restriction site. The ubiquitin mutant K48R was created by exchanging the lysine codon (AAG) with a codon for arginine (CGT) using the Quick Change mutagenesis kit from Stratagene (La Jolla, Calif., USA). The ubiquitin variant Ub-SAC with the C-terminal extension Ser-Ala-Cys was generated by PCR with C-terminal primers encoding the three additional amino acids. The correctness of the plasmids was confirmed by double-stranded DNA-sequencing with at least 2-fold overlap and the sequences analysed with the Phred-Phrap software (Applied Biosystems, Foster City Calif., USA). UCH-L3 was subcloned from the library plasmid with GenBank N27190 by PCR into the EcoRI/XhoI sites of pGEX-4TI (Amersham) encoding for a GST-UCH-L3 fusion protein. For cloning of USP2 (USP2 isoform 2; Swiss Prot 075604) a cDNA clone containing full length USP2a (MGC: 1315 IMAGE:3543435) was used to amplify different parts via PCR. The PCR reactions were performed with ProofStart DNA Polymerase (Qiagen, Hildesheim, Germany) in 30 cycles with 20 sec at 94° C., 20 sec at 62° C. and 60 sec at 72° C. The catalytic domain only (USP2 core) was amplified, cloned into pCR2.1-TOPO (TOPO TA Cloning; Invitrogen 45-0641). The insert was further subcloned NcoI (partial)/NotI into a modified version of the vector pET15b (Novagen) which contains a StrepTag behind a NotI site. The resulting plasmid pET15b-hu USP2 core-ST is coding for the core domain of USP2 fused to a C-terminal StrepTag via an AAA linker. The isoform 2 of USP2 (USP2 short) was amplified, cloned into pCR2.1-TOPO (TOPO TA Cloning; Invitrogen). The insert was further subcloned NdeI (partial)/XhoI into pET24a (Novagen) to get pET24a-hu USP2short. To get an expression plasmid for isoform 2 with a C-terminal Strep Tag for purification the tagged C-terminus of pET15b-hu USP2core-ST was subcloned BamHI/XhoI into pET24a-hu USP2 short. The insert of the resulting plasmid pET24a-hu USP-2 short-ST has been controlled by sequencing. The insert has the expected changes at the N-terminus but no further variations compared to Genbank entry BC002955.

1.2 Protein Expression and Purification

For the production of ubiquitin (Ub) and the four ubiquitin conjugating enzymes, cells of the E. coli expression strain BL21 (DE3) Tuner (Novagen) were transformed with the corresponding plasmids. For expression cells were grown at 37° C. in Terrific Broth (TB, complemented with 0.1 M MOPS buffer pH 7.0) in 2 liter shaker flasks at 200 rpm to an OD₆₀₀ of 0.8. After induction with 0.1 mM isopropylthiogalactoside (IPTG) the cells were further incubated for 16 hours at 20° C. and harvested by centrifugation. The cells were lysed by 2-fold passage on a French Press (Thermo Spectronic), whereby 0.5 mM Pefabloc® (Roche, Basel, Switzerland) and 50 U/ml benzonase (Merck, Darmstadt, Germany) were added. The tagged fusion proteins (Trx-Ub, NusA-Ub, His₆-Cdc34, His₆-E2-25K, Trx-Rad6B and Trx-UbcH10) were purified over a Ni-NTA Superflow (Qiagen) column installed on a ÄKTA 100 Explorer (Amersham Biosciences, Freiburg, Germany) chromatography system. After loading of the lysates the column was washed with 5 column volumes of binding buffer (50 mM Na-phosphate, 300 mM NaCl, 20 mM imidazole pH 8.0) and the His₆-tag containing fusion proteins were eluted by increasing the concentration of imidazole to 300 mM. The pooled fractions containing the target protein were either directly used (His₆-Cdc34, His₆-E2-25K) or further processed by cleaving of the tag with PreScission protease (Amersham Biosciences) overnight at 4° C. Removal of the tag and second purification was achieved by anion-exchange chromatography (Resource Q, 6 ml) with cleaved Trx-Ub, Trx and Trx-UbcH10. Both proteins were found in the flow-through when a buffer of 20 mM Tris-HCl pH 7.5 was used, while the tag and the contaminants still bound to the column. In the case of Trx-Rad6B the tag was separated by a second Ni-NTA column on which the cleaved non-tagged E2 enzyme appeared in the flow-through. NusA-Ub was further purified on a Superdex 75 (XK26/60) size exclusion column with 1×PBS as buffer. All purified proteins were characterized by mass spectrometry, whereby in all cases the preparations contained the protein with the correct mass in a purity >90%. Ubiquitin activating enzyme E1 was obtained from elsewhere. The expression plasmid for UCH-L3 was transformed into E. coli strain BL21(DE3)pLysS which was cultivated in LB medium containing 100 μg/ml ampicillin and 34 μg/ml chloramphenicol and was induced at OD₆₀₀ of 0.5 with 0.5 mM IPTG. After 5 hours of induction the cells were harvested by centrifugation. All purification steps were done at 4° C., unless stated otherwise. Cells from 4 liter E. coli cell culture were resuspended in PBS buffer at pH 7.3 containing 1% PMSF and 2 mM DTE and ruptured by sonication (4 times 30 seconds at 60% amplitude; Branson Digital Sonifier W-450D). After centrifugation of the homogenate at 75000 g for 15 min, the supernatant was applied to a glutathione sepharose column (GSTPrep FF 16/10, Amersham) equilibrated with PBS at a flow rate of 2 ml/min. After washing with 3 column volumes, UCH-L3 was eluted at a flow rate of 3 ml/min with PBS supplemented with 10 mM GSH. Fractions were analyzed by SDS-PAGE (4-20%) and the UCH-L3 containing fractions were pooled and concentrated to about 10 ml. Immediately after concentration, the sample was applied to a size exclusion chromatography column (Superdex 75, HiLoad 26/60, Amersham) equilibrated with PBS to avoid extended exposure of GSH to the protein. The GST-fusion tag was removed by incubating the sample for 1 week with thrombin (10U/mg protein) at 10° C. After the incubation, the sample was applied to a glutathione sepharose column (GSTPrep FF 16/10, Amersham) to remove the remaining undigested fusion protein. The flow-through containing thrombin and mature UCH-L3 was applied to a size exclusion chromatography column (Superdex 75, HiLoad 26/60, Amersham) equilibrated with buffer C (10 mM Tris, 100 mM NaCl, pH 8.0) at a flow rate of 2.5 ml/min. The UCH-L3 containing fractions were pooled, dropped into liquid nitrogen and stored at −80° C. For the production of USP2 isoform 2 cells of the E. coli expression strain BL21(DE3) pLysS (Novagen, Darmstadt, Germany) were transformed with the plasmid pET24a-hu USP2 short-ST and was cultivated in LB medium containing 30 μg/ml kanamycin, 34 μg/ml chloramphenicol and induced at OD₆₀₀ of 0.5 with 0.5 mM IPTG. After 5 hours of induction the cells were harvested by centrifugation. All purification steps were done at 4° C., unless stated otherwise. Cells from 2 liter E. coli cell culture were resuspended in 25 mM Tris/HCl, pH 8.5, supplemented by 100 mM NaCl, 2 mM DTE and ruptured by sonication (2 times 30 seconds at 60% amplitude; Branson Digital Sonifier W-450D). After centrifugation of the homogenate at 75000 g for 15 min, the supernatant was applied to a Strep-Tactin column (150/10, IBA, Goettingen, Germany) equilibrated with buffer A (25 mM Tris/HCL, 100 mM NaCl, pH 8.5) at a flow rate of 2 ml/min. After washing with 3 column volumes, USP2 was eluted at a flow rate of 1 ml/min with buffer B (buffer A supplemented with 2,5 mM desthiobiotin pH 8.0). Fractions were analyzed by SDS-PAGE (4-20%) and the USP2 containing fractions were pooled and concentrated to approximately 18 ml. The sample was applied to a size exclusion chromatography column (Superdex 75, HiLoad 26/60, Amersham) equilibrated with 10 mM Tris/HCL, 100 mM NaCl, pH 8.0 at a flow rate of 2.5 ml/min. The USP2 containing fractions were pooled, dropped into liquid nitrogen and stored at −80° C.

### 1.3 Chemical Labeling of Ub-SAC

A site-specific and chemical labeling was performed on the additional C-terminal cysteine residue of the ubiquitin variant with the C-terminal Ser-Ala-Cys extension (Ub-SAC) with maleinimide fluorophores such like TAMRA, Cy5. In order to reduce the protein, which could form dimers because of the C-terminal cysteine, Ub-SAC solution was incubated with 1 mM EDTA and 20 mM DTT, respectively. Before labeling, the protein was dialysed overnight against 1×PBS pH 7.4 to eliminate the previous additives which interfere with the chemical modification of the cysteine residue with the fluorophore. Stock solutions of TAMRA and Cy5 maleinimide labels, commercially available were dissolved in dimethylsulfoxide. For each labeling experiment, a two-fold excess of fluorophore was incubated for three hours at room temperature with Ub-SAC. The latter was only present in the mix at 30% since the C-terminal extension Ser-Ala-Cys had been cleaved during expressing and Ub in the native form was present as the major component. The labeled product was then separated from the excess of fluorophore by a desalting step on a PD10 column (Sephadex G 25M, Amersham Biotech) and fractions containing labeled Ub-SAC were detected using reverse phase HPLC. Since the fractions pool still contained a mixture of unlabeled Ub and labeled Ub-SAC, a semi-preparative RP-HPLC was performed on a C4 Vydac (Dionex) column allowing the purification of the desired labeled product.

### 1.4 Synthesis of Labeled Peptide and Lysine

FMOC-LIFAGK(BOC)Q(Trt)LE(tBu)D(Trt)G peptide bound C-terminally to TentaGel TG-SRAM (initial loading of the first amino acid 0.2 mmol/g) was synthesized by (Jerini A G, Berlin, Germany). FMOC-Lys(BOC)—OH residue (Novabiochem) was coupled with one repetition to Rink amide resin (100-200 mesh, Novabiochem, initial loading 0.5 mmol/g). The peptide and the lysine residue were labeled by modified standard protocols according to the Fmoc/tBu chemistry in filterable vessels. One equivalent of peptide or lysine residue bound to the synthesis resin was treated with 20% piperidine/DMF (1, 3, 10 minutes) to remove the protective FMOC-group, washed multiple times with DMF and methylenechloride and reacted overnight with 3 equivalents of 6-carboxytetramethylrhodamine (Molecular Probes), which was pre-activated for 10 minutes with 3 equivalents of 2-(1H-Benzotriazole-1-yl)-1,1,3,3-tetramethyluronium hexafluorophosphate and N-hydroxybenzotriazole (Novabiochem) in the presence of 3 equivalents of diisopropylethylamine. After extensive washing of the resin with DMF and methylenechloride, cleavage of the labeled peptide and lysine from the resin and full de-protection was performed with trifluoroacetic acid/triethylsilane/water (95:3:2) in 1.5 h. The solvent was removed, the residuals were re-dissolved in few μl trifluoroethanol and precipitated with diethylether. The precipitation procedure was repeated twice. Final purification of the products was achieved by preparative HPLC on Nucleosil 5 C18 PPN with a linear gradient of acetonitrile (0.08% TFA)/0.1% TFA from 15:85 to 60:40 in 40 min at a flow rate of 5 ml/min. Purity and integrity of the products TAMRA-LIFAGKQLEDG-NH₂ and TAMRA-Lys-NH₂ was verified my LC-MS analysis.

### 1.5 Enzymatic Labeling of Ub-K48R

For the enzymatic conjugation of fluorescent labels to the C-terminal COOH group of ubiquitin the various components were mixed together as follows: 200 μM of native ubiquitin (bovine red blood cells; SIGMA) or recombinant proteins Ub-K48R, Trx-Ub K48R, 100 μM NusA-Ub K48R, 20 μM of one of the ubiquitin conjugating enzymes E2, 0.2 μM ubiquitin activating enzyme E1 and 1 mM of the lysine fluorophores attached to the a amino groups of lysine or the undecapeptide L-1-F-A-G-K-Q-L-E-Q-G-NH₂. The reaction volumes ranged between 0.1 and 5 ml and the reaction buffer consisted of 50 mM HEPES, 50 mM NaCl, 5 mM MgCl₂, 4 mM ATP and 0.2 mM DTT. The whole mixture was incubated for four hours at 37° C. and in some cases further overnight at 25° C. The progress of the reaction was monitored by SDS-PAGE gel electrophoresis with NuPAGE gels (Invitrogen, Carlsbad Calif., USA) and by reversed phase HPLC on a Poros R1/10 column (Applied Biosystems, Foster City Calif., USA). The modified ubiquitin derivatives were again purified in order to remove the other reagents like E1, E2 and free fluorophores. Ub and NusA-Ub were purified on size exclusion columns (Superdex 75 HR30/10 or Superdex 200 XK16/60, Amersham Biosciences), whereby in the case of Ub labeled with the TAMRA-decapeptide an additional separation step was performed onto a Vydac C4 (Dionex, Sunnyvale Calif., USA) reversed phase column. Labeled Trx-Ub was purified on a 1 ml Ni-chelate Hi-Trap (Amersham Biosciences) on which the His₆-tag containing Ub derivative was bound specifically.

### EXAMPLE 2

Assay Development and Substrates Evaluation

The assay is based on a change in fluorescence polarization and lifetime due to the cleavage of the de-ubiquitinases, between a short labeled peptidic sequence and the ubiquitin linked to it. The different ubiquitin variants tested in this project consist either of a Ser-Ala-Cys-Dye C-terminal extension of ubiquitin or fluorescent lysine derivatives coupled to the C-terminal COOH group of ubiquitin through an isopeptide bond. While the processed substrates (cleaved fluorescent extentions) show a lower polarization and lifetime, the unprocessed C-terminally labeled ubiquitins give higher fluorescence polarization and lifetime values.

Measurements of polarization and lifetime are performed on the research reader of Evotec OAI (see below). To perform the test, the de-ubiquitinating enzyme is first put into the well and the addition of the substrate triggered the start of the reaction. The enzymatic reaction is stopped by shifting the pH from 7.5 to 5 with the addition of Stop buffer (end concentration of 30 mM malonic acid pH 4.5—see TABLE 1). With this procedure the read-out could be kept stable for several hours.

TABLE 1
Assay buffers compositions

| | References |
|---|---|
| Enzyme and substrate dilution buffer | |
| TRIS - 20 mM - pH 7.5 | SIGMA #T-6666 |
| NaCl - 100 mM | Fluka #71380 |
| EDTA - 1 mM | Fluka #03677 |
| DTT - 5 mM | Applichem #A1101,0025 |
| CHAPS - 0.1% | Applichem #A1099,0050 |
| BSA - 0.05% | SIGMA #A-3059 |
| Stop buffer | |
| Malonic acid - 180 mM - pH 4.5 | Lancaster #4227 |

Several C-terminally labeled Ub variants are synthesized and assayed as DUB substrates (TABLE 2). NusA-His6-S-Ub-K48R-lysine-TAMRA, resulting from the modification of a large tagged Ub with α-NH2-TAMRA-lysine, gave the highest dynamic range (table 1) in terms of fluorescence polarization. This substrate is cleaved by UCH-L3 and USP2, and is therefore used for the development of the HTS assay.

TABLE 2
Summary of the dynamic range values obtained in de-ubiquitination experiments
with various C-terminally labeled ubiquitin substrates.

| | Lifetime | Lifetime | Delta (ns) | Delta (ns) | Polarization | Polarization | Delta (mP) | |
|---|---|---|---|---|---|---|---|---|
| | Uncleaved | cleaved | 100% | 50% | Uncleaved | cleaved | 100% | Delta (mP) |
| SUBSTRATES | (ns) | (ns) | conversion | conversion* | (mP) | (mP) | conversion | 50% conversion* |
| Ub-SAC-TAMRA | — | — | — | — | 160 | 50 | 110 | 55 |
| Ub-SAC-Cy5 | 1.4 | 0.95 | 0.45 | 0.225 | 310 | 200 | 110 | 55 |
| Ub-K48R-Peptide-TAMRA | — | — | — | — | 160 | 70 | 90 | 45 |
| Ub-K48R-lysine-TAMRA | — | — | — | — | 165 | 50 | 110 | 55 |
| Trx-His₆-S-Ub-K48R-lysine- | — | — | — | — | 220 | 50 | 170 | 85 |
| TAMRA | | | | | | | | |
| Trx-His₆-S-Ub-K48R-lysine-Cy5 | 1.54 | 1.1 | 0.44 | 0.22 | 310 | 180 | 130 | 65 |
| NusA-His₆-S-Ub-K48R-lysine- | — | — | — | — | 250 | 50 | 200 | 100 |
| TAMRA | | | | | | | | |

Both enzymes, USP-2 and UCH-L3 are found to give identical results for total conversion of the substrate (absolute dynamic range). Highest sensitivity of the assay is found at 50% substrate conversion. Therefore the dynamic range relevant for the HTS is calculated for half maximal substrate conversion (*). As mentioned, changes in fluorescent polarization, but not fluorescent lifetime (for Cy5 derivatives) are selected as an activity measure for AD and HTS.

NusA-His6-S-Ub-K48R-lysine-TAMRA is identified as the best substrate for USP-2 and UCH-L3, we varied the enzyme concentration and followed the reaction over time. As expected, conversion of the substrate is found to be enzyme concentration and time dependent.

### 2.1 Deubiquitinases Assay Read Out

The confocal optics of the Evotec Olympus Research Reader are adjusted with TAMRA and the G factor is determined knowing the true polarization value for free TAMRA (34 mP). Thus the cleavage of the large NusA-His₆-S-Ub-K48R-lysine-TAMRA (250 mP) by USP-2 or UCH-L3 into a short TAMRA-peptide (50 mP) and NusA-His₆-S-Ub is followed by measuring polarization. This readout allowed us also to determine the affinity as well as the velocity of the deubiquitinases for this substrate as shown in the next paragraph.

### 2.2 Determination of Michaelis Constants by Means of Fluorescence Polarization

In order to use polarization values (P) to determine the Michaelis-Menten constants, P has to be converted into convenient physical parameters such as molar concentration of processed (Pt) or unprocessed substrate (St).

As shown previously, polarization can be expressed as:

$$P = \frac{3 {P}_{b} \times Y - {P}_{f} ( 3 Y + {P}_{b} - 3 )}{( 3 + Pb \times ( - 1 + y ) - ( 3 + ( - 3 + Pf ) ) \times y )}$$

Where

### P: polarization measured

### Pb: polarization in 100% bound state (here unprocessed substrate)

### Pf: polarization in 100% free state (here 100% processed substrate)

### S₀: total amount of substrate in the reaction (M)

### Sₜ: amount of unprocessed substrate (M)

### Pₜ: amount of processed substrate

Therefore

$$Y = \frac{( Pf - P ) \times ( 3 - Pb )}{( P - 3 ) \times ( Pb - Pf )}$$

Where

$$Y = \frac{{S}_{t}}{{S}_{0}} and {S}_{0} = {S}_{t} + {P}_{t}$$

Thus

$${P}_{t} = {S}_{0} \times ( 1 - \frac{( Pf - P ) \times ( 3 - Pb )}{( P - 3 ) \times ( Pb - Pf )} )$$

### 2.3 Results

### 2.3.1 Kinetic Study for USP-2

In order to determine the K_{M} and k_{cat} of the deubiquitinases for NusA-His₆-S-Ub-K48R-lysine-TAMRA we used different substrate concentrations and followed the reaction by fluorescence polarization. These measurements are performed on a Perkin-Elmer LS-55 spectrometer which allows direct injection of substrate or enzyme into a stirred cuvette for immediate measurement after the reaction has been started.

The resulting data are transformed according to the formula described below and plotted displaying the product formed on the y-axis (Pt) and time on the x-axis. As these transformed data represent an absolute (product formation) and not a ratiometric signal (polarization), the slope of the curves should increase in proportion to the substrate concentration. At high substrate concentration however this was found not to be the case. Therefore these results point to substrate inhibition of USP-2 at elevated substrate concentrations.

From these data, the rates of initial velocity are calculated and used to determine maximal velocity Vₘₐₓ and the affinity constant K_{M} of USP-2 for the substrate NusA-His₆-S-Ub-K48R-lysine-TAMRA using the following equation:

$${v}_{i} = \frac{{V}_{max} \times {S}_{0}}{Km + {S}_{0}}$$

Where

vᵢ: Initial velocity

### Vₘₐₓ: maximal velocity (k_{cat}·E)

### K_{M}: Michaelis Constant

### S₀: total substrate concentration

Analogous experiments are performed for the deubiquitinase UCH-L3. As this enzyme was found to be ten to twenty times more active than USP-2, very low amounts of UCH-L3 (or highly diluted protein solutions) had to be dispensed in kinetic experiments. This caused significant variations of the data and consequently led to imprecise values. Since the kinetic characterization of the DUBs is not of central importance for the development of the HTS, we decided not to include these data into this report.

### 2.3.2 Inhibition of the Deubiquitinases

Cysteine proteases, such as USP-2 and UCH-L3, are sensitive to covalent modification of the active site cystein. As compounds acting covalently on this key residue of the enzyme do not represent pharmacologically interesting lead candidates, successful HTS assays need to selectively identify non-covalent inhibitors. Ubiquitin aldehyde is a known inhibitor for both enzymes, USP-2 and UCH-L3. However, as Ubiquitin aldehyde forms a covalent inactive intermediate with the active site cysteine of the enzymes, this molecule can only be used as a reference compound. In absence of reducing agent this inhibitor is known to be very potent (pM activity range). Using this molecule we analyzed whether addition of DTT can be utilized to mask the activity of covalent active site inhibitors during the screen. When titrating Ubiquitin aldehyde in presence of 5 mM DTT an IC₅₀ value of 100 nM was found. The shift of the IC₅₀ from a pM range to 100 nM indicates that (at least some) covalent modifiers will not show up as false positives during the screen.

### 2.3.3 Assay Window and Downscaling

### 2.3.3.1 Assay Window

The sensitivity of a given HTS assay is most crucial for the identification of not only very potent, but also moderate or even weekly active inhibitors. A mathematical simulation can be used to predict the behavior of potential inhibitors in the assay. Based on the enzymatic constants, determined earlier during characterization of the enzymatic activity, we simulated the effect of competitive inhibitors with various hypothetical affinities (Ki from 10 nM to 5 μM) for USP-2 (no simulation for HCH-L3 could be run, since kcat and Km are not determined). The Km of USP2 for NusA-His₆-S-Ub-K48R-lysine-TAMRA was previously determined to be 150 nM and kcat to be 0.15 s⁻¹. As the substrate concentration in the assay is far below Km (10 nM), substrate consumption over time can be described by the equation for a single turnover reaction mechanism:

$$S ( t ) = {S}_{0} \times {e}^{- ( \frac{{k}_{cat}}{Km} \times Ext )}$$

In the presence of a competitive inhibitor 1, substrate consumption can be expressed as:

$$S ( t ) = {S}_{0} \times {e}^{- ( \frac{{k}_{cat}}{Km ( 1 + \frac{I}{Ki} )} \times Ext )} \qquad ( 1 )$$

where I is the concentration of the inhibitor.

As shown previously, polarization values are converted using equations (2) and (3):

$$\begin{matrix}Y = \frac{{S}_{t}}{{S}_{0}} and & ( 2 ) \\ P = \frac{3 {P}_{b} \times Y - {P}_{f} ( 3 Y + {P}_{b} - 3 )}{( 3 + Pb \times ( - 1 + y ) - ( 3 + ( - 3 + Pf ) ) \times y )} & ( 3 )\end{matrix}$$

By combining these three equations we are now able to predict the effect of a given competitive inhibitor in a dose dependent manner for any hypothetical Ki. Under HTS conditions, where test compounds are assayed at a final concentration of 20 μM, the present assay is expected to detect inhibitors with Kᵢ values of up to (or even above) 10 μM. If an arbitrary threshold of 40% inhibition of the polarization signal is selected, 20 μM inhibitors will be picked up by the screen.

### 2.3.3.2 Downscaling

One important factor which has to be tested during downscaling of the assay to the 1 ml level (2080 well nanoplates) is the assay's compatibility with hydroxy beta cyclodextrin (HBC) and DMSO. Nanocarriers are coated with HBC in order facilitate re-solubilization of dried test compounds (‘dry screening’) and to reduce adsorption of assay components to the carrier. If HBC interferes with the assay, pre-coated plates can not be used and ‘wet screening’, utilizing compounds dissolved in DMSO, has to be performed. When adding increasing concentrations of DMSO to the assay, only a minimal interference with the reaction was noticed. Even DMSO concentrations of up to 5% are found to be compatible with the screen. When adding HBC, a reduction of the enzymatic activity was observed. These findings support a ‘wet screening’ format for these target family.

A second goal of downscaling is to preserve the quality of the signal and the dynamic range of the assay when miniaturizing from 20 ml to the final assay volume. No change in enzymatic activity was observed when performing the assay in 2080 well nanocarriers. Equally, the dynamic range of the assay was comparable with data generated in assay development plates. Additionally, the assay reagents have shown to be stable over several hours in the nanodispenser reservoirs. Taken together these data show that the assay is robust enough to be implemented on the NanoScreening platform.

We claim:

1. A method for measuring a deubiquitination activity comprising combining a deubiquitinating enzyme (DUB) with a substrate comprising a ubiquitin moiety and a fluorescently labeled compound under conditions allowing for deubiquitinating activity and measuring an altered fluorescence polarization and/or fluorescence lifetime of the released fluorescently labeled compound.

2. A method according to claim 1 wherein the fluorescently labeled compound is an amino acid or a peptide with a fluorophore.

3. A method according to claim 1 wherein the amino acid is a lysine or lysine derivative with a fluorophore.

4. A method according to claim 1 wherein the peptide comprises at least one lysine residue and has 2 to 50 amino acid residues.

5. A method according to claim 1 wherein the fluorescently labeled compound is linked via an isopeptide bond to the C-terminus of the ubiquitin moiety of the substrate.

6. A method according to claim 1 wherein the fluorescently labeled compound is an amino acid or peptide having 2 to 50 amino acid residues with a fluorophore attached to the C-terminus of the ubiquitin moiety via a peptide bond.

7. A method according to claim 1 wherein the ratio of the molecular mass of the substrate to the free fluorescently labeled compound is at least 2.

8. A method according to claim 7 wherein the fluorescently labeled compound is attached enzymatically to the ubiquitin moiety.

9. A method according to claim 8 wherein the fluorophore of the fluorescently labeled compound is TAMRA, Cy3, Cy5, MR121 or EvoBlue.

10. A method according to claim 1 wherein specific lysine residues of the ubiquitin moiety have been mutated to arginine like amino acids.

11. A method according to claim 1 wherein the DUB is UCH-L3 or USP2 or any other DUB such as USP5, USP6, USP4, USP8, USP13, USP2, USP11, USP14, USP7, USP9X, USP10, USP1, USP12, USP16, USP15, USP17, USP19, USP20, USP3, USP9Y, USP18, USP21, USP22, USP33, USP29, USP25, USP36, USP32, USP26, USP24, USP42, USP46, USP37, USP28, USP47, USP38, USP44, USP50, USP35, USP30, Mername-AA088peptidase, Mername-AA091 peptidase, USP45, USP51, USP34, USP48, USP40, USP31, Mername-AA129peptidase, USP49, USP17-like peptidase, USP54, USP53, USP39, UCH-L1, UCH-L3, UCH-BAP1, UCH-UCH37, Cezanne deubiquitinating peptidase, Cezanne2, tumor necrosis factor alpha-induced protein 3, TRABID protein, VCP(p97)/p47-interacting protein, otubain1, otubain2, CylD protein, SENP1 peptidase, SENP3 peptidase, SENP6 peptidase, SENP2 peptidase, SENP5peptidase, SENP7peptidase, SENP8peptidase, SENP4peptidase, Poh1 peptidase, Jab1/MPN domain metalloenzyme, Mername-AA 165 peptidase, Mername-AA 166 peptidase, Mername-AA167 peptidase, Mername-AA168 protein, COP9 signalosome subunit6, 26S proteasome non-ATPase regulatory subunit7, eukaryotic translation initiation factor3 subunit5, IFP38 peptidase homologue.

12. A method of screening for a modulating agent of a DUB comprising measuring deubiquitination activity according to claim 1.

13. A method according to claim 12 wherein the activity of the deubiquitinating enzyme is inhibited.

14. A method according to claim 1 wherein the method is performed in a High Throughput Screening (HTS) platform.

15. A substrate library comprising peptides of the formula (Z)ₐ(X)ₘK(X)ₙ(Z)_{b} wherein X can be any amino acid except Lys wherein Z is distinct amino acid which is used to increase solubility preferably a charged amino acid, more preferably an Arg and wherein a and b are independently an integer from 0 to 5, and n and m are independently an integer from 0 to 20 provided that not both n and m are 0 and wherein K is attached to the C-terminus of a ubiquitin moiety via an isopeptide bond.

16. A method to identify the substrate specificity of a DUB comprising reacting the DUB with a substrate library of claim 15.
